(19) United States
(12) Patent Application Publication
(10) Pub. No.: US 2013/0295195 A1
(43) Pub. Date: Nov. 7, 2013

(54) AQUEOUS ADDITIVE FOR POULTRY WATER

(71) Applicant: CONTACT MARKETING SOLUTIONS INNOVATIVE TECHNOLOGIES, Danbury, CT (US)

(72) Inventors: Jerry B. Creasey, Conroe, TX (US); Jason H. Creasey, Conroe, TX (US); David H. Creasey, Splendora, TX (US)

(73) Assignee: Contact Marketing Solutions Innovative Technologies, Danbury, CT (US)

(21) Appl. No.: 13/795,820

(22) Filed: Mar. 12, 2013

Related U.S. Application Data
(60) Provisional application No. 61/641,449, filed on May 2, 2012.

(51) Int. Cl. A23K 1/175; A61K 33/34; A61K 33/38

(52) U.S. Cl. CPC A23K 1/175; A61K 33/38; A61K 33/34
USPC 424/618; 424/637

(57) ABSTRACT

A method for increasing weight gain, water consumption, and rate of food consumption in poultry by administering an aqueous, acidic composition to the poultry drinking water is presented.

### RELATED APPLICATIONS

This application claims priority to U.S. Provisional Application No. 61/641,449, which was filed on May 2, 2012, and is incorporated herein by reference in its entirety for all purposes.

### FIELD OF THE INVENTION

The invention relates to methods for increasing meat yield in poultry. More specifically, this invention relates to methods for increasing meat weight and yield in commercially grown poultry such as chickens and turkeys by administration of effective amounts of an aqueous composition that improves weight gain in poultry.

### BACKGROUND

Poultry production has become increasingly competitive as mergers result in fewer poultry companies throughout the world. However, per capita poultry consumption continues to increase worldwide, and poultry breeders must grow their products faster and provide more yield of the most valuable parts of the carcass, i.e., muscle. Accordingly, there is a need in the commercial poultry industry for methods that more rapidly increase muscle weight gain in poultry.

### SUMMARY OF THE INVENTION

A method is provided for increasing muscle weight gain and general health in poultry. The method comprises administering to the poultry an aqueous composition comprising an acid selected from the group consisting of sulfuric acid, phosphoric acid, fumaric acid, acetic acid and hydrochloric acid; at least one of an ammonium compound, sodium sulfate, potassium sulfate, and magnesium sulfate; and at least one metal ion selected from the group consisting of copper, silver, zinc, magnesium, manganese, nickel and iron, wherein the metal ion is provided as a metal sulfate; wherein consumption of the composition increases weight gain in the poultry.

### BRIEF DESCRIPTION OF THE DRAWINGS

FIG. 1 is a graph showing the average daily weight of chicks receiving untreated water (control) or water containing the additive.

### DETAILED DESCRIPTION OF THE INVENTION

An acidic composition of matter is disclosed in U.S. Pat. Nos. 5,989,595 and RE 41,109 to Cummins, which are incorporated herein by reference in their entirety. As described below, it has now been found that adding a modification of this composition to the water supply of young poultry improves growth rate, weight gain, and general health of the poultry. The increase in weight gain is believed to be due, in part, to increased food consumption in birds receiving the composition.

The composition is aqueous and comprises, as a first component, an acid selected from sulfuric acid, phosphoric acid, fumaric acid, acetic acid, nitric acid or hydrochloric acid. The acid is preferably of high purity, i.e., between about 89 percent to about 99.9 percent purity. A second component is selected from an ammonium compound, sodium sulfate, potassium sulfate, and magnesium sulfate. The ammonium compound is preferably anhydrous ammonia, ammonia monohydrate, ammonium sulfate, or urea ammonium nitrate (UAN), most preferably, ammonium sulfate. The composition further comprises at least one metal ion. The metal ion is preferably selected from copper, silver, zinc, magnesium, manganese, nickel, and iron. Other noble metals and titanium could also be used. The metal ion is generally provided as a metal sulfate.

In one embodiment, the second component of the composition is prepared by first heating water of any type, preferably distilled or deionized water, to between 65° F. and 210° F., preferably 70° F. to 170° F. The ammonium compound is added to the heated water to give a concentration of about 5% to about 35% and the solution is mixed to dissolve the ammonium compound. In other embodiments, sodium sulfate, potassium sulfate, or magnesium sulfate is combined with the water instead of an ammonium compound. In another embodiment, the second component is added to water that has not been heated.

The resulting solution of the second component is then added simultaneously with the acid to a pressure vessel. The acid is added to comprise about 10% to about 60% of the final concentrated solution, preferably about 15% to 40%. In one embodiment, the reaction is allowed to proceed under pressure of atmospheric to about 80 psi above atmospheric pressure, preferably between atmospheric and about 15 psi above atmospheric pressure, while a DC current is passed through the mixture at a current of at least one amp. In other embodiments the application of pressure above atmospheric and/or DC current is omitted.

The time and temperature of the reaction will vary based on the amount of reactants, size of reactor and reactivity of selected reactants. The temperature of the mixture is maintained in a range between about 125° F. and about 1000° F. during the reaction. The reaction time varies between 30 minutes and 6 hours, preferably from about 1 to about 3 hours. A cooling jacket is required to control the temperature of the reaction. After the reaction has proceeded to completion, the mixture is allowed to cool or can be cooled using any appropriate means. In one embodiment, the original solution of the second component is added to about 10% to about 15% of the total weight of the final, cooled mixture. In another embodiment, this step is omitted.

The pH of the final, concentrated solution is zero or less. The concentrated solution is diluted with water, preferably distilled or deionized water, and additional components are added. One or more metal sulfates are added in a concentration range of about 0.5% to about 30%. Mixing of the components can be performed by any appropriate means. The composition is compatible with additives such as vitamin supplements, minerals, and therapeutic compounds. The acid composition is diluted in a range of from about 1:10 to about 1:5000.

The diluted composition is added to water or to a watering system to provide treated water to poultry as described in Example 1. The composition is added to the poultry water at about 0.2 to about 20 ounces per gallon of water, preferably about 0.3 to about 15 ounces per gallon. The concentration of the composition in the water may be increased as the age and/or size of the birds increases. The composition may be provided to any type of commercially raised, including free-range, poultry, including chickens, turkeys, ducks, geese, and other fowl.

### EXAMPLES

### 1. Effect of Aqueous Additive on Growth Rate and Muscle Weight in Broiler Chickens

Aqueous, acidic compositions containing ammonium sulfate or sodium sulfate were prepared as described above. Diluted solutions containing metal sulfates were prepared as shown in Table 1.

TABLE 1

| Composition | Sodium Sulfate (ml) | Ammonium Sulfate (ml) |
|---|---|---|
| Water | 100 | 100 |
| Acid composition | 60 | 60 |
| *CuSO4 soln | 50 | 50 |
| *AgSO4 soln | 20.5 | 0 |
| Final volume | 230.5 | 210 |

Treated water was delivered by an on demand watering system in a ratio of 1 ounce diluted composition per gallon of water. The pH of treated water was approximately 6.4 and the pH of untreated water was approximately 7.0. The final concentration of copper sulfate in the water for each additive was 10 ppm. The final concentration of silver sulfate in the water containing the sodium sulfate additive was 3 ppb.

Twenty, two week old chicks were housed in enclosed pens at 10 chicks per pen, (1 ft²/chick) with free access to a high protein starter feed mix containing 17% protein and free access to water. For 10 days, a first pen of 10 chicks received untreated water (control) and a second pen of 10 chicks received water containing the sodium sulfate composition of Table 1 (treated). After 10 days, the sodium sulfate additive was replaced by the ammonium sulfate additive of Table 1 (treated). Water containing the ammonium sulfate composition was provided for 46 days. Chicks were weighed, generally, at two day intervals.

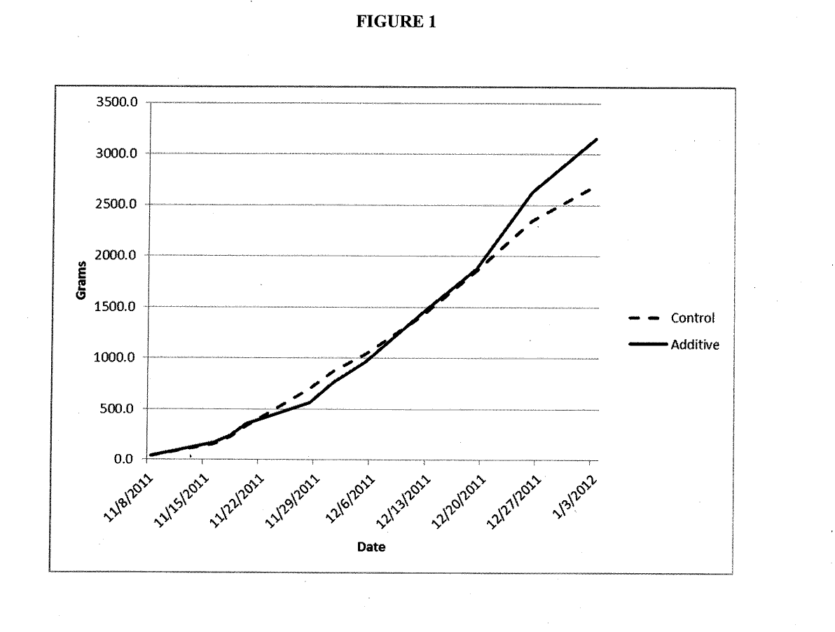

Results are shown in FIG. 1. Statistical analysis (Welch two independent samples t-test) demonstrated a highly significant difference (p=0.003) in weight gain of the treated and control birds over the 56 days of the experiment. The median percent increase in weight gain obtained by using the test treatment versus the control is 17%, with a 95% confidence interval of 6% to 30%.

At the conclusion of the experiment, the birds were killed and processed to remove heads, feet, feathers, and organs. Processed carcasses were weighed. Control processed birds averaged 2106 g and treated processed birds averaged 2390 g in weight. Visual inspection indicated an increase in breast and leg size in the treated birds.

During the course of the experiment, the estimated food consumption rate of birds receiving treated water showed a greater increase over time (increase from 2 to 3.5 lbs/day over 46 days) than the estimated food consumption rate of birds receiving untreated water (2 lbs/day, stable over 46 days). In addition, birds receiving treated water also consumed more water (0.93 L/bird/day) than birds receiving untreated water (0.68 L/bird/day), based on measurements of water remaining in the watering system at the end of the experiment. These results suggest that the composition could be used to administer therapeutic additives to poultry more efficiently.

As shown in FIG. 1, birds receiving the treated water showed more extensive weight gain than birds receiving untreated water. After processing, carcasses of birds receiving treated water were, on average, 284 g (13%) heavier than birds receiving untreated water.

In addition, the birds receiving treated water showed an improvement in general health compared with control birds, e.g., reduced foot swelling, indicating improved immune function, and improved respiratory function.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

We claim:

1. A method for increasing weight gain in poultry comprising providing an aqueous composition comprising an acid selected from the group consisting of sulfuric acid, phosphoric acid, fumaric acid, acetic acid, nitric acid and hydrochloric acid; at least one of an ammonium compound, sodium sulfate, potassium sulfate, and magnesium sulfate; and at least one metal ion selected from the group consisting of copper, silver, zinc, magnesium, manganese, nickel and iron, wherein the metal ion is provided as a metal sulfate; and administering the composition to the poultry, wherein consumption of the composition increases weight gain in the poultry.

2. The method of claim 1, wherein the ammonium compound of the composition is selected from the group consisting of anhydrous ammonia, ammonium sulfate, ammonium nitrate, and urea ammonium nitrate.

3. The method of claim 1, wherein the at least one metal ion is present at a concentration of 0.5% to 30%.

4. The method of claim 1, wherein the aqueous composition is administered to the poultry in the poultry water supply.

5. The method of claim 1, wherein the aqueous composition added at a final concentration of 0.2 to 20 liquid ounces of the composition per gallon of water.

6. The method of claim 1, wherein the composition comprises sulfuric acid, ammonium sulfate, and at least one metal ion selected from copper and silver.

7. The method of claim 1, wherein providing the aqueous composition further comprises
(a) preparing an aqueous, concentrated acidic solution by the steps comprising
i) combining in a pressurized or non-pressurized vessel an acid of at least 89% purity, selected from the group consisting of sulfuric acid, phosphoric acid, fumaric acid, acetic acid, nitric acid and hydrochloric acid, with water and at least one of an ammonium compound, sodium sulfate, potassium sulfate, and magnesium sulfate to provide a mixture;
ii) maintaining the temperature of the mixture of step i) in a range from 125° F. to 1000° F. and allowing the components of the mixture to react;
iii) cooling the mixture or allowing the mixture to cool;
(b) diluting the mixture with water; and
(c) adding a selected amount of at least one metal ion to the solution of step (b), wherein the metal ion is provided as a metal sulfate.

8. The method of claim 7, wherein the ammonium compound of the composition is selected from the group consisting of anhydrous ammonia, ammonium sulfate, ammonium nitrate and urea ammonium nitrate.

9. The method of claim 7, wherein the metal sulfate is added to a concentration of 0.5% to 30%.

10. A method for increasing the rate of food consumption in poultry comprising administering to the poultry an aqueous composition comprising an acid selected from the group consisting of sulfuric acid, phosphoric acid, fumaric acid, acetic acid, nitric acid and hydrochloric acid; at least one of an ammonium compound, sodium sulfate, potassium sulfate, and magnesium sulfate; and at least one metal ion selected from the group consisting of copper, silver, zinc, magnesium, manganese, nickel and iron, wherein the metal ion is provided as a metal sulfate; and administering the composition to the poultry, wherein consumption of the composition increases the rate of food consumption by the poultry.

11. A method for increasing the rate of water consumption in poultry comprising administering to the poultry an aqueous composition comprising an acid selected from the group consisting of sulfuric acid, phosphoric acid, fumaric acid, acetic acid, nitric acid and hydrochloric acid; at least one of an ammonium compound, sodium sulfate, potassium sulfate, and magnesium sulfate; and at least one metal ion selected from the group consisting of copper, silver, zinc, magnesium, manganese, nickel and iron, wherein the metal ion is provided as a metal sulfate; and administering the composition to the poultry, wherein consumption of the composition increases the rate of water consumption by the poultry.
